(19) United States
(12) Patent Application Publication
(10) Pub. No.: US 2005/0094101 A1
(43) Pub. Date: May 5, 2005

(54) Method and apparatus for measuring the effect of different lighting conditions

(76) Inventor: Ian Jordan, Cambridgeshire (GB)

Correspondence Address: BIRCH STEWART KOLASCH & BIRCH, PO BOX 747, FALLS CHURCH, VA, 22040-0747, US

(21) Appl. No.: 10/946,619

(22) Filed: Sep. 22, 2004

Related U.S. Application Data
(63) Continuation-in-part of application No. 10/464,491, filed on Jun. 19, 2003.
Application No. 10/464,491 is a continuation-in-part of application No. PCT/GB03/01362, filed on Mar. 28, 2003.

(30) Foreign Application Priority Data
Dec. 21, 2000 (GB) 0031384.1
Nov. 30, 2001 (GB) 0128705.1
Mar. 28, 2002 (GB) 0207303.9

(51) Int. Cl. A61B 3/10

(52) U.S. Cl. 351/221

(57) ABSTRACT

A light box (10) with a viewing aperture contains sources of illumination (22, 28) which can be adjusted and set to simulate different lighting situations and conditions. An observable image on a trolley (40) is movable towards and away from the observer (14) and also transversely to the line of sight of the observer. Movement is effected by stepping motors (36 and 38). Measurements are made of the response of the observer to movement of the image consequent on changes in the parameters affecting the observer's perception of the image. A comparative assessment can thus be made of the effect of changing the illumination.

### CROSS REFERENCE OF THE RELATED APPLICATION

This application is a Continuation-In-Part of copending PCT application Ser. No. 10/464,491 filed on Jun. 19, 2003, the entire contents of which are hereby incorporated by reference and for which priority is claimed under 35 U.S.C. § 120. In addition, Applicant claims that this Continuation-In-Part Application also claims priority of Application No. PCT/GB03/01362 filed on Mar. 28, 2003 under 35 U.S.C. § 120, the entire contents of which are hereby incorporated by reference.

### FIELD OF THE INVENTION

This invention relates to methods of and apparatus for the testing and assessment of eyesight defects and medical conditions associated with eyesight. It is more particularly concerned with methods of and apparatus for measuring changes in the human response to changes in lighting and/or the movement of images.

Various medical conditions such as dyslexia, nystagmus, squint and migraine are affected by light conditions, i.e. by one's environment.

Although various methods of testing such conditions are known, there is currently no method of or apparatus for measuring the effect of changes in lighting conditions. It is known for example that dyslexia is affected by the brightness of the ambient light and the colour of the ambient light. It is also known that tinted lenses can be used to assist in the correction of squint in order to restore the correct muscle balance. It is also known that certain medical conditions such as migraine can be triggered by the flicker from cathode ray tubes.

### SUMMARY OF THE INVENTION

It is an object of the present invention to provide methods of and apparatus for testing the response of an observer to different lighting conditions and from those tests obtaining measurements which will assist in treating or correcting these conditions.

It is a further object of the invention to provide methods of and apparatus for simulating different conditions of illumination, for example daylight, tungsten, fluorescent and CRT flicker.

With the invention one can make quantitative measurements of the ability of an observer to perceive images which may be either still or moving, and which are presented in a controlled illumination environment, with the aim of ascertaining whether the perception of the observer is enhanced by changes in the lighting parameters. The degree of that enhancement can also be determined. In a preferred embodiment, quantitative measurements can be made of the degree of enhancement by the interposition of differently coloured lenses or filters. The results relating to the different lighting situations can be used to improve a person's clarity of perception by choosing appropriate lens tints to suit those particular situations.

In accordance with the invention there is provided optical assessment apparatus comprising: i) a housing providing with at least one viewing aperture through which an observer is arranged to look; ii) means defining an observable image within the housing; iii) illumination means arranged to provide illumination of the image; iv) first control means operative to vary the illumination of the image; v) second control means arranged to effect movement of the image; and, vi) means to measure and/or record the response of the observer to movement of the image consequent on changes in the parameters affecting the observer's perception of the image.

The apparatus of the present invention preferably comprises a light box within which an image, for example a dot, is viewed by the observer, with the lighting conditions within the light box simulating those of a particular real-life situation. In tests where the image is designed to move, the perpendicular distance of the plane of the image from the observer is measured, so that the observer can make judgements as to the clarity or integrity of the image at different distances from the eye. Also, the speed and direction of movement of the image in a plane perpendicular to the line of sight of the observer may be varied.

Also in accordance with the invention there is provided a method of assessing eyesight comprising viewing an observable image within a housing, providing illumination of the image, moving the image within the housing, and measuring and/or recording the response of an observer to movement of the image consequent on changes in the parameters affecting the observer's perception of the image.

All the information or data relating to these parameters can be collected and stored to give a record of each test.

The colour temperature of the lighting used to illuminate the image may be altered. to simulate different real-life situations of illumination. Within each lighting mode which is chosen, the intensity of the individual components of the total lighting may be adjusted and/or the frequency of the flicker of the lighting can be chosen to simulate various real-life situations. The apparatus preferably includes one or more optical filters or other devices which can be interposed between the eye of the observer and the image, to allow the effect of the filters to be recorded and differences noted. In a situation where the image is moving, it can be moved at different speeds both towards and away from the observer and transversely to the line of sight of the observer.

The image which is viewed by the observer can be viewed with incident light or alternatively one can arrange for the image to be displayed on a transparent or translucent background to simulate the monitor screen of a cathode ray tube. The light source simulating the CRT may be modulated in intensity and/or in flicker frequency and/or in waveform to create different illumination effects. In this configuration, the image may simultaneously be lit by incident light as well as by the back lighting.

By use of the invention one can actually measure the effect of changes in the tint of tinted lenses so that one can determine the optimum tint to use for any particular condition.

In the case of sunglasses for example, one can optimise the tint for each individual.

In the case of photochromic lenses, four or five different types of tint are currently used. Most of these use broad spectrum absorption dyes. By use of the apparatus of the present invention one can test the observer with different tints and the apparatus will show the degree of improvement, or the degree of worsening, achieved for each lens.

The apparatus of the present invention is particularly useful in assessing the effect of flicker, either from fluorescent lighting or from VDUs. One can set the flicker within the light box to a particular frequency and then change that frequency, obtaining measurements for each frequency.

For example, for those who suffer from migraine as a result of flicker, one can simulate different types of CRT, operating at different frequencies, and from this predict which screens are most likely to trigger the problem. One thus has a means of non-invasive therapy by creating a job environment which is the least harmful to any particular individual.

### BRIEF DESCRIPTION OF THE DRAWINGS

In order that the invention may be more fully understood, one presently preferred embodiment of apparatus in accordance with the invention will now be described by way of example and with reference to the accompanying drawings. In the drawings:

FIG. 1 is a schematic plan view of an optical assessment apparatus in accordance with the invention, with the cover removed;

FIG. 2 is a schematic side elevation showing the disposition of the lamps positioned at the sides of the apparatus;

FIG. 3 is a front view of the trolley and slide guide of the apparatus of FIG. 1;

FIG. 4 is a plan view of the trolley and slide guide shown in FIG. 3;

FIG. 5 is a front view of the trolley showing the mounting of the pendulum;

FIG. 6 is a plan view showing how the pendulum is driven from the pendulum motor;

FIG. 7 is a plan view of the apparatus, omitting some of the features shown in FIG. 1, but illustrating additional features; and,

FIG. 8 is a front elevation of a belt drive arrangement associated with the motion of the pendulum.

### DESCRIPTION OF THE PREFERRED EMBODIMENT

The optical assessment apparatus of the present invention is essentially a light box, and will hereinafter be referred to as such. In the drawings the light box is indicated generally at 10. It comprises a housing 12 from which extraneous light is excluded. An observer, represented by eyes 14, is positioned at the front end of the light box and views the interior of the light box through an aperture or apertures in the front end wall 16 of the housing. The aperture or apertures can be in the form of a slot or slots.

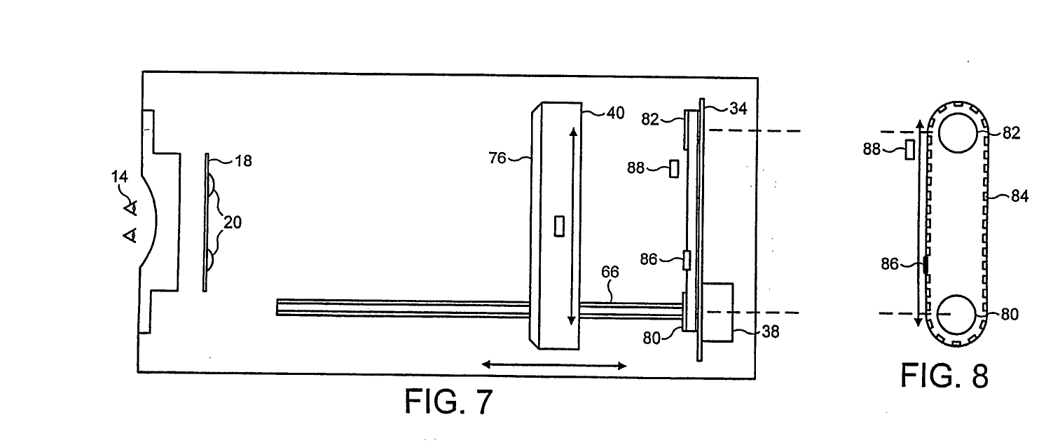

As shown in FIG. 7, a holder 18 can be mounted within the light box adjacent to the viewing slots, to receive lenses or filters 20. These are positioned in alignment with the eye slots. Preferably, the holder 18 has provision for the receipt of two lenses or filters in a side-by-side arrangement. Alternatively, the holder 18 could be in the form of a rotatable carousel, so that different lenses or filters can be brought selectively into alignment with the eye-line of the observer. The lenses or filters could be a fixed series of lenses or filters, or alternatively could be introduced selectively by the tester. As yet a further alternative, lenses or filters 20 could be introduced into openings in the front end wall 16 of the housing itself.

At the end of the light box 10 remote from the observer is a light source 22 which is preferably a D-lamp. Positioned in front of the lamp 22 is a diffuser panel 24. A coloured filter panel 26 is positioned in front of the diffuser panel 24. At each side of the housing 12 there is mounted a set of light sources 28 in the form of tubes. In the illustrated embodiment, four light tubes 28 are provided one above another on each side of the light box. Diffuser panels 30 are positioned in front of the light tubes 28 to diffuse the light which is transmitted into the interior of the light box.

These side diffuser panels can be removable. The light tubes 28 are not all the same. They are arranged to provide for illumination which represents daylight, light from tungsten filaments and fluorescent light. By switching appropriately between the tubes one can thus create conditions within the light box which simulate daylight, tungsten-filament light or fluorescent light. Control means are also provided to enable the brilliance of the lamps 22 and 28 to be adjusted, for example in ten discrete steps. This provides a further means of varying the illumination within the light box. The control circuitry for the light box is housed within the base of the housing and is indicated at 29.

Extending lengthwise of the housing, from adjacent to the end remote from the observer to the end adjacent to the observer, is a slide guide 32. Fixedly mounted on the slide guide 32, towards the end of the light box remote from the observer, is a bulkhead plate 34. This is positioned symmetrically in relation to the slide guide 32. The diffuser panel 24, the filter panel 26 and the bulkhead plate 34 are in practice located close together. They are shown separated for clarity. The bulkhead plate 34 carries two stepping motors 36 and 38. Stepping motor 36 is a travel′motor which is used to effect displacement of a trolley 40 lengthwise within the light box, as indicated by arrow 42. A lead screw 44 extends from travel motor 36 towards the front end 16 of the housing and passes through a female nut 46 towards the base of the trolley 40. Rotation of the lead screw 44 by the travel motor 36 interacts with the female nut 46 to cause longitudinal displacement of the trolley 40 towards and away from the observer.

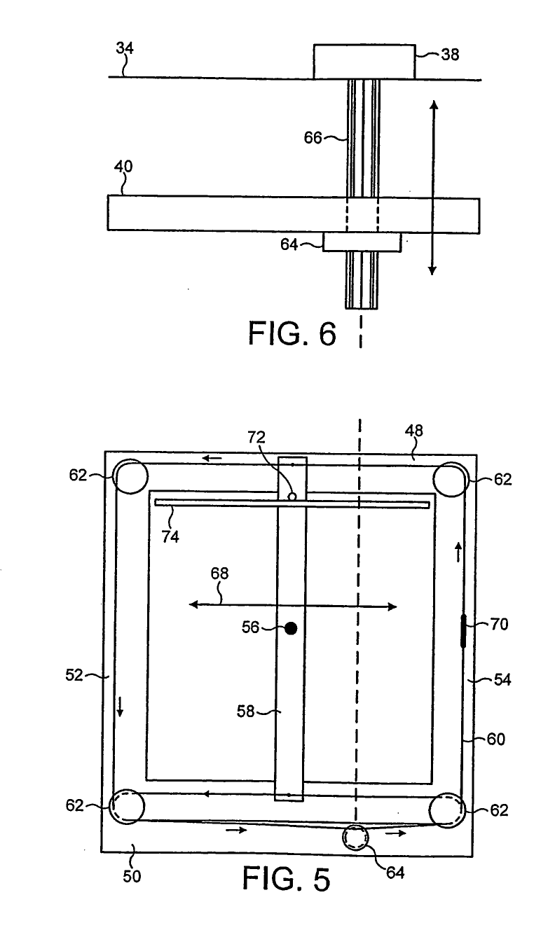

The displaceable trolley 40 is essentially a carrier for an image which is to be viewed by the observer in order to enable an assessment to be made of the observer's eyesight under different conditions. This is best illustrated by reference to FIG. 5. The trolley 40 essentially comprises an open-centred plate having a top frame member 48, a bottom frame member 50 which carries the female nut 46 and side frame members 52 and 54. The female nut 46 and lead screw 44 are not shown in FIG. 5 for clarity. The image which is viewed by the observer is indicated at 56. This image can be just a spot, or can be a symbol. The image 56 is provided on what is hereinafter referred to as a pendulum 58. The pendulum 58 is a vertically arranged strip of plastics material which bridges the gap between the top frame member 48 of the trolley and the bottom frame member 50. The pendulum 58 is suspended from a pendulum transmission cord 60 which extends through holes at the top and bottom of the pendulum and which passes around four grooved pulleys 62 which are positioned one at each corner of the trolley frame. The cord 60 is tangential to each pulley. At the bottom of the trolley there is provided a splined pulley 64 which meshes with a splined shaft 66 which is rotated by stepping motor38 mounted on the bulkhead 34. As shown in FIG. 6, the splined pulley 64 is mounted on the side of the trolley bottom frame member 50 which faces towards the observer. The transmission cord 60 passes around the splined pulley 64 which is rotationally driven from the stepping motor 38 by way of the splined shaft 66. The transmission cord is looped around the pulleys 62, 64 so that on rotation of the splined shaft 66 the pendulum 58 will traverse back and forth across the width of the trolley, as indicated by arrow 68. A spring 70 is incorporated in the transmission cord 60 in order to provide adequate tension in the cord. Towards the upper end of the pendulum 58 it is provided with a rolling wheel 72 which rests upon a horizontal guide rail 74 which extends substantially the full width of the trolley. The wheel 72 and guide rail 74 provide stability for the pendulum as it traverses the trolley. Instead of using splined shaft 66 one could alternatively use a hexagonal cross-section bar.

Actuation of the pendulum motor 38 will thus cause the pendulum 58 to traverse, to the left, or to the right or back and forth. Preferably, control means are provided whereby the pendulum can move at a number of different traverse speeds, for example three traverse speeds. Control means can also be provided so that the pendulum will oscillate back and forth across the trolley at any one of a plurality of different speeds, for example with a traverse time of one second, two seconds or four seconds. The control means can also function so that the pendulum will“stall”in its centre position and/or at its extreme positions to left and right.

The image 56 which is provided on the pendulum 58 is preferably a spot which is viewed by the observer. However, one could alternatively use symbols, writing or pictures.

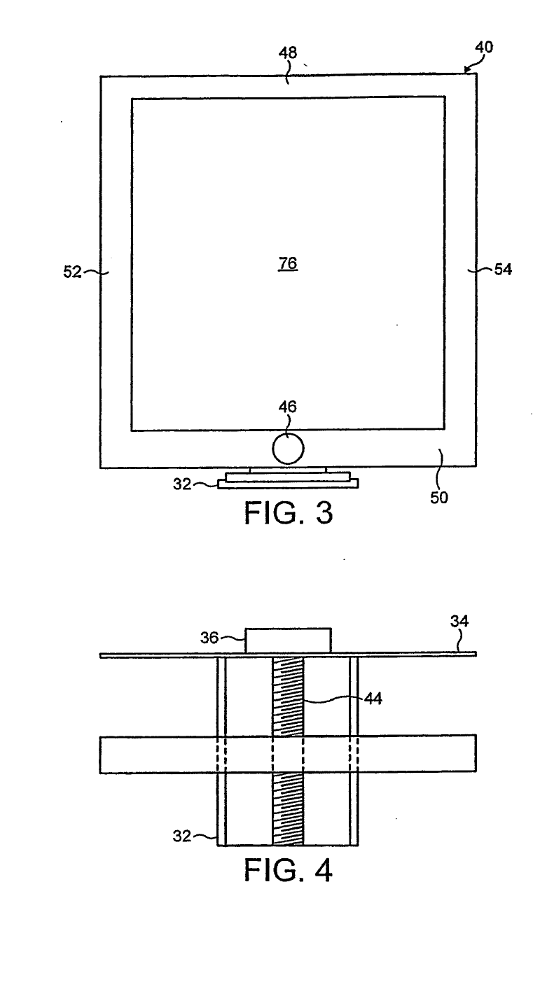

As shown in FIGS. 3 and 7, a plain screen 76 can be fitted at the front of the trolley 40. If opaque, this can be used to blank off the image 56 by covering the pendulum.

Its area is also available for writing, patterns, etc. The screen could alternatively be transparent. The screen 76 can slide down between side channels provided on the trolley, and when it is removed enables the image to be seen.

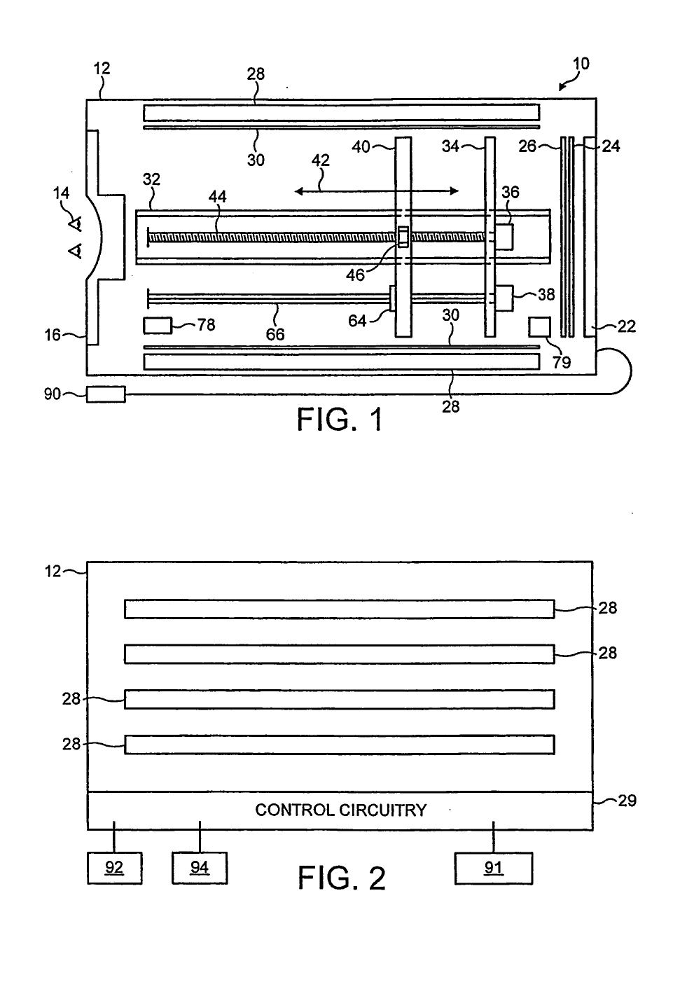

As shown in FIG. 1, a forward travel detector 78, e.g. a photo-detector, is provided to limit displacement of the trolley 40 towards the observer. As the trolley reaches this detector 78 switch means are actuated to prevent further forward movement. An equivalent detector 79 is provided to limit displacement of the trolley 40 away from the observer.

As shown in FIGS. 7 and 8, means are provided for controlling and detecting the motion of the pendulum 58 as explained above. The stepping motor 38 effects a traversing movement of the pendulum by way of the splined shaft 66, the splined pulley 64 and the pendulum transmission cord 60.

Mounted on the front of the bulkhead 34 are two pulleys, a drive pulley 80 and an idler pulley 82. A toothed belt 84 travels around these pulleys. The toothed belt 84 carries a flag 86 which projects up from the belt. Positioned in front of the belt 84 is a pendulum end travel detector 88, for example a photo-detector. The end travel detector 88 is positioned to define the limit of travel of the pendulum 58 in one direction across the width of the trolley. When the pendulum motor 38 is operating, to traverse the pendulum, it will also cause the belt 84 to travel around the pulleys 80 and 82. When the flag 86 which is carried by the belt 84 reaches the pendulum end travel detector 88, the flag will be detected and the detector will cause the pendulum motor 38 to stop, reverse its motion, and thus cause the pendulum to traverse in the opposite direction, to the other end of its limit of travel. The pendulum stepping motor 38 is linked to appropriate counting means in the control circuitry so that one can determine the setting of the pendulum, and hence of the image 56, from the number of traverse steps which the motor has effected.

The light sources 22 and 28 are such as to enable the creation of different forms of illumination of the image 56 within the light box, to simulate different real-life situations of illumination. By selecting different light sources and varying them, the intensity of the individual components of the total lighting may be adjusted and/or the frequency of the flicker of the lighting can be adjusted to simulate various real-life situations. By a suitable choice of light sources and by appropriate adjustment, one can simulate daylight conditions, tungsten lighting or fluorescent lighting. By varying the power supply to the light sources one can vary the intensity of illumination. In order to simulate office conditions with fluorescent lighting one can introduce flicker, for example at 100 Hz for the United Kingdom and at 35 Hz for the USA.

Using the light sources 28, the image 56 would be illuminated with incident light. By using the light source 22 at the end of the light box the image can be back-lit, in order to simulate a monitor screen of a cathode ray tube for example. The light source 22 which simulates the cathode ray tube can be modulated both in intensity and/or in flicker frequency to create different illumination effects. The flicker frequency can be 50,87.5 or 110 Hz for example.

The provision of the light sources 22 and 28 and the means of adjustment of these sources means that one can recreate various environments, for example a person's job environment. Having created a particular environment one can test the observer by monitoring their observation of the image 56 as it is moved or changed within the light box.

What the apparatus does in essence is to simulate particular lighting conditions, thereby to establish a base line. Then, by changing the lighting conditions and/or any filters and/or the motion of the image 56, one can carry out various test procedures on the observer and obtain comparative measurements in response to these changes.

In use, what one is primarily concerned to measure is the distance between the eye of the observer and the image 56.

It is from this measurement that one can obtain comparative results consequent on movement of the image 56 towards and away from the observer and transversely across the line of sight of the observer. The image 56 is moveable in these two dimensions. In use, the tester will arrange for movement of the trolley 40 and/or of the pendulum 58, under appropriately set lighting conditions. The observer will view the image and will signal, using a remote button 90 (FIG. 1), when the image 56 appears or disappears as the case may be. In order to obtain a measurement of the position of the image 56 lengthwise of the light box, the travel motor 36 is arranged to drive the trolley 40 first to one limit detector 78 or to the other 79. Using the stepping motor one can then count back from that end detector to determine the position of the image when the observer presses the remote button 90 to signal the appearance or disappearance of the image. This procedure can be operated in the advancing or receding mode of the trolley.

In addition to the primary measurement, i.e. the distance of the image 56 from the observer, one also can measure the position of the image relative to the centre line of the light box, using the output from the pendulum stepping motor 38.

From these measurements one can obtain comparative test results to enable the eyesight of the observer to be assessed.

Although one is concerned with the measurement of distances, the control circuitry is arranged automatically to convert these distance measurements to dioptres, so that a measurement-is presented for assessment purposes.

Although not shown in the drawings, the light box is provided with a plastics moulded cover on top of the housing.

This incorporates a keyboard 91, a touch screen 92 and a printer 94 linked to the control circuitry 29 in the base of the light box. By using the printer 94 one can obtain a visual record of the test procedure. The testing of the observer by the tester can be controlled by the measurements and observations made by the tester. Alternatively, an alphanumeric keyboard and display can be used. The tester will select certain tests by a menu which will prompt the sequence of events comprising the test. The tester can choose inter alia the types of lighting, the lighting levels, the introduction of flicker, the speed and direction of movement of the image and the filters used. The operating sequence is controlled by a microprocessor which operates in accordance with a software routine. The apparatus will not only store this data but will also store information on the response of the observer in relation to a particular test or sequence of tests. All this data can then be printed so that comparisons can be made and in particular one can actually measure the effect of changing the test parameters. One can thus establish that there is an improvement in certain optical phenomena by changing various parameters, but one can also measure improvement and degree of improvement. As mentioned above, the observer preferably indicates his satisfaction with the observation of an image by using the remote button 90.

The signal generated by the remote button is used in conjunction with monitoring equipment as part of the data input to the test procedure. By appropriate use of the remote button the observer can indicate when the image is visible/invisible, changes, is clear/unclear, etc. This information is then added to the other data which is recorded at the same time, so that an analysis of the observations can subsequently be carried out.

In an alternative embodiment of the apparatus, a light box is again used, but the image is displayed on a strip of flexible material. This material may be either opaque or light-transmitting and could also be variable along its length in respect of this property. The strip is wound on a pair of spools, one at each side of the light box. Either both spools can be driven or alternatively one can be driven with the other relying on stored energy, for example from a spring.

The spools and their associated mechanism are fixed to the body of the light box. The strip of flexible material wound on the two spools is passed over a further set of spools which are mounted on a moving carriage. The carriage is biased by a force or forces in such a manner as to apply tension to the flexible strip, for example by means of springs. When the spools are driven in the same direction and at the same speed, the effect is to allow the strip to move in a fixed plane. If the spools are driven in opposite directions and at the same speed then the effect is to allow the plane of the strip to move forwards or backwards relative to the observer. By rotating the spools at different speeds, a combined effect can be obtained where the plane either recedes or advances at the same time as it allows the image to move within the plane of the strip.

To increase the range of images which are available, the strip could be part of an interchangeable cassette of images, for example in different languages, to allow easy extension of the flexibility of the apparatus. As a further option, the movable carriage could be fitted with a locating device to allow the insertion of a fixed which could be either opaque or transparent.

The spring or springs providing the tension to the strip are arranged so that they are offset in such a manner that the effects of any tolerances in the mechanism are eliminated by causing the carriage to run along a single guiding face and not between two faces. The strip can be provided with markers along its length so that its position can be monitored to allow re-zeroing calibration to take place during operation or at the end of each test.

1. Optical assessment apparatus comprising:
a housing provided with at least one viewing aperture through which an observer is arranged to look;
means defining an observable image within the housing;
illumination means arranged to provide illumination of the image;
first control means operative to vary the illumination of the image;
second control means arranged to effect movement of the image; and
means to measure and/or record the response of the observer to movement of the image consequent on changes in the parameters affecting the observer's perception of the image.

2. Apparatus as claimed in claim 1, in which the illumination means comprises at least two different light sources.

3. Apparatus as claimed in claim 1, in which the illumination means comprises means to produce daylight conditions and/or tungsten light conditions and/or fluorescent light conditions within the housing.

4. Apparatus as claimed in claim 1, in which the intensity of the illumination can be varied.

5. Apparatus as claimed in claim 1, in which measurements are made of the distance between the observer's eye and the image.

6. Apparatus as claimed in claim 1, in which the image is displaceable towards and away from the observer and/or transversely to the line of sight of the observer.

7. Apparatus as claimed in claim 6, in which the displacement of the image is controlled by a stepping motor.

8. Apparatus as claimed in claim 1, in which the image is presented as a viewing target on a strip of material carried by support means and displaceable relative to the support means by rotation of a drive shaft connected to a stepping motor.

9. Apparatus as claimed in claim 8, in which the strip is suspended by a cord arranged to be moved by motion of the drive shaft to cause transverse movement of the strip between predetermined limits to each side of the line of sight of the observer.

10. Apparatus as claimed in claim 1, in which the image is carried by a trolley displaceable towards and away from the observer between limit positions by stepping motor means and a shaft connecting the stepping motor means to the trolley.

11. Apparatus as claimed in claim 1, which includes means for detecting the position of the image transversely to the line of sight of the observer, and arranged to reverse the transverse motion of the image at a traverse limit position.

12. Apparatus as claimed in claim 11, in which the detecting means comprises a photoelectric detector positioned at said limit position, a continuous belt driven by the motor means causing traverse of the image, and a detector flag carried by the belt and arranged to be sensed by the detector at said limit position.

13. Apparatus as claimed in claim 1, in which the measurement of the distance between the image and the eye of the observer is determined using stepping motor means arranged to provide a count from an end limit position.

14. Apparatus as claimed in claim 1, in which the image is a spot.

15. Apparatus as claimed in claim 1, which includes means to introduce one or more filters into the line of sight of the observer.

16. Apparatus as claimed in claim 1, which includes means to introduce flicker to the light produced by the illumination means.

17. A method of assessing eyesight comprising:
providing an observable image within a housing;
providing illumination of the image;
moving the image within the housing; and
measuring and/or recording the response of an observer to the movement of the image consequent on changes in the parameters affecting the observer's perception of the image.

18. A method as claimed in claim 17, which comprises moving the image towards and away from the observer and measuring and/or recording the distance between the eye of the observer and the image.

19. A method as claimed in claim 17, which includes moving the image transversely to the line of sight of the observer.

20. A method as claimed in any of claims 17, which includes mounting the image on a carrier which is displaceable towards and away from the observer, and measuring the distance of the image from the eye-of the observer by measuring the position of the carrier relative to an end limit position thereof.
